(19) United States
(12) Patent Application Publication
(10) Pub. No.: US 2022/0391919 A1
(43) Pub. Date: Dec. 8, 2022

(54) METHOD AND APPARATUS FOR WARNING A USER THAT A REPORT MAY BE LACKING INFORMATION

(71) Applicant: MOTOROLA SOLUTIONS, INC., Chicago (IL)

(72) Inventors: CHARIS ENHUI WEE, Bayan Lepas (MY); WEI KEONG LOOI, Bayan Lepas (MY); DANNY CHEW CHIAN HONG, Bayan Baru (MY); CLEMENT PAKIAM TOBIAS XAVIER, Batu Ferringhi (MY)

(21) Appl. No.: 17/303,787

(22) Filed: Jun. 8, 2021

(51) Int. Cl. G06Q 30/00; G08B 21/18; G06F 40/279; G06Q 10/10; G16H 40/67; G06Q 50/26

(52) U.S. Cl. CPC G06Q 30/018; G08B 21/18; G06F 40/279; G06Q 10/10; G16H 40/67; G06Q 50/26

(57) ABSTRACT

A method and apparatus for notifying authors of potential missing information within in a report is described herein. During operation, an activity level of a personal-area network (PAN) assigned to the author is monitored over time. Additionally, the author's report is analyzed to determine a content score over time that indicates an amount of report content for various time periods. If, for a particular time period, an activity level of the PAN greatly exceeds the content score of the report, the author is warned about potential missing content. As part of the warning to the person, a summary of activity of the PAN activity may be provided to the author to remind them of potential missed content within the report.

### BACKGROUND OF THE INVENTION

As part of a first responder's duties, various documentations such as reports and forms need to be filled out by first responders after certain incidents. Due to time constraints, many of the documentations cannot be filled in their entirety while at a particular incident, leaving the remainder of the documentation to be filled at a later time. This often leads to officers often forgetting information. It would be beneficial if an officer can be prompted when it is believed that the officer has left out information within a report.

### BRIEF DESCRIPTION OF THE SEVERAL VIEWS OF THE DRAWINGS

The accompanying figures where like reference numerals refer to identical or functionally similar elements throughout the separate views, and which together with the detailed description below are incorporated in and form part of the specification, serve to further illustrate various embodiments and to explain various principles and advantages all in accordance with the present invention.

FIG. 1 illustrates an operational environment for the present invention.

FIG. 2 depicts an example communication system that incorporates a personal-area network and a digital assistant.

FIG. 3 is a more-detailed view of a personal-area network of FIG. 2.

FIG. 4 is a block diagram of a report server.

FIG. 5 is a graph illustrating an activity score and a content score for an officer.

FIG. 6 is a block diagram of server 401.

FIG. 7 is a flow chart showing operation of the server of FIG. 6.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention. It will further be appreciated that certain actions and/or steps may be described or depicted in a particular order of occurrence while those skilled in the art will understand that such specificity with respect to sequence is not actually required.

### DETAILED DESCRIPTION

In order to address the above-mentioned need, a method and apparatus for notifying authors of potential missing information within in a report is described herein. During operation, an activity level of a personal-area network (PAN) assigned to the author is monitored over time. Additionally, the author's report is analyzed to determine an amount of report content existing for various time periods. If, for a particular time period, an activity level of the PAN greatly exceeds the amount of content within the report, the author is warned about potential missing content. As part of the warning to the officer, a summary of PAN activity for the time may period be provided to the author to remind them of potential missed content within the report.

Consider the following example: An officer was part of a foot chase that involved the officer abruptly stopping and drawing their weapon at 7:50 PM. The officer wrote a report having very little mentioned as happening at 7:50 PM. Because of this, a report server will notify the officer that at 7:50 PM an accelerometer detected an abrupt stop and a gun draw sensor detected a gun draw event, and that the report includes little information around the 7:50 PM time period.

Prior to describing the above concept in detail, the following definitions are provided:

Active Sensor— A sensor is determined to be “active” when the sensor is detecting an event that exceed a particular threshold for that sensor. For example, an accelerometer may be active for a time period where it has detected an acceleration of over 0.1 g, a gun-draw detector may be active for a time period during which it has detected a gun has been drawn, a velocity detector may be active during a time period where it has detected a velocity of over 3 meters per second . . . etc.

Activity Score— A numerical value that represents a number of active sensors for a particular period of time. A higher score preferably represents more sensor activity than a lower score. For example, if two sensors are determined to be active (i.e., above a sensor threshold) between 7:50 PM and 7:55 PM, then an activity score of 2 may be assigned to that time period, where if four sensors are determined to be active between 7:50 PM and 7:55 PM, then an activity score of 4 may be assigned to that time period.

Content Score—An amount of content within a report describing events that takes place over a particular time period. A higher number for a time period preferably represents more content describing what happened over that time period. The content score may be something as simple as a number identifying how many words were written to describe what happened during that time period. So, for example, if 50 words were written to describe what happened between 7:50 PM and 7:55 PM, a content score of 50 may be generated for that time period, while if 100 words were written to describe what happened between 7:50 PM and 7:55 PM, a content score of 100 may be generated for that time period. In alternate embodiments, natural-language processing may be performed on the report to determine actual content/intent/topics of what was written. When this takes place, the content score may be based on a number of topics discussed during that time period. So, for example, if two topics (e.g., running, and drawing a weapon) are discussed that took place between 7:50 PM and 7:55 PM, a content score of 2 may be generated for that time period, while if three topics (e.g., running, fighting, and drawing a weapon) are discussed that took place between 7:50 PM and 7:55 PM, a content score of 3 may be assigned to this time period.

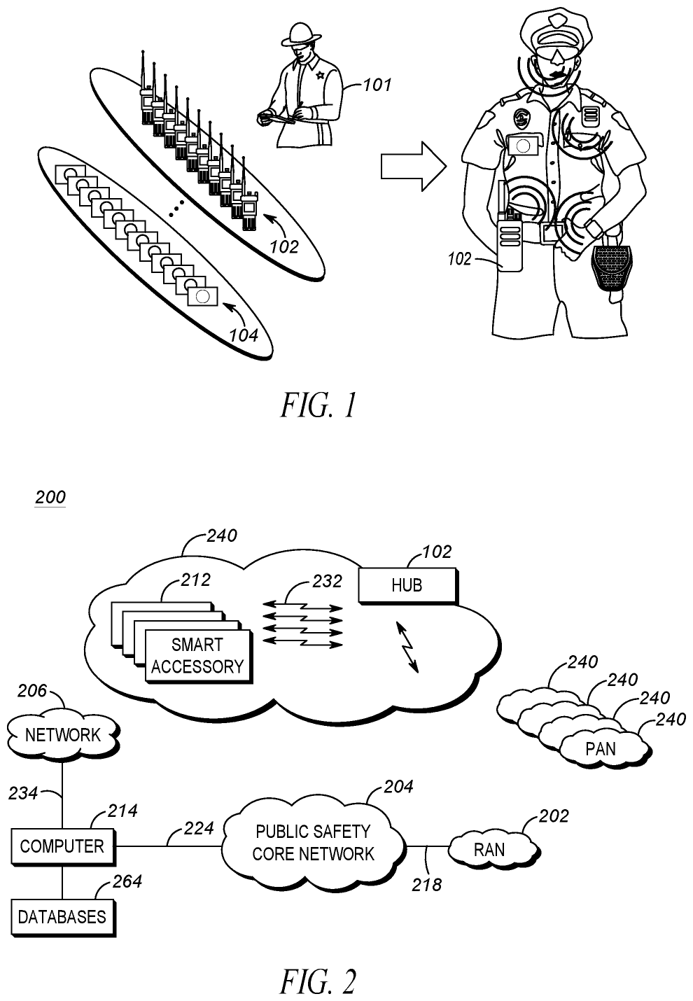

Turning now to the drawings, wherein like numerals designate like components, FIG. 1 illustrates an operational environment for the present invention. As shown, a public safety officer 101 will be equipped with devices that determine various physical and environmental conditions surrounding the public-safety officer. These conditions are generally reported back to a dispatch center so an appropriate action may be taken. For example, future police officers may have a sensor that determines when a gun is drawn. Upon detecting that an officer has drawn their gun, a notification may be sent back to the dispatch operator so that, for example, other officers in the area may be notified of the situation.

It is envisioned that the public-safety officer will have an array of shelved devices available to the officer at the beginning of a shift. The officer may select some of the devices off the shelf, and form a personal area network (PAN) with the devices that will accompany the officer on his shift. For example, the officer may acquire a gun-draw sensor, a body-worn camera, a wireless microphone, a smart watch, a police radio, smart handcuffs, a man-down sensor, a bio-sensor, . . . , etc. All devices acquired by the officer will be configured to form a PAN by associating (pairing) with each other and communicating wirelessly among the devices. The PAN comprises more than two devices, so that many devices are connected via the PAN simultaneously.

A method called bonding is typically used for recognizing specific devices and thus enabling control over which devices are allowed to connect to each other when forming the PAN. Once bonded, devices then can establish a connection without user intervention. A bond is created through a process called “pairing”. The pairing process is typically triggered by a specific request by the user to create a bond from a user via a user interface on the device.

As shown in FIG. 1, public-safety officer 101 has an array of devices to use during the officer's shift. For example, the officer may acquire one radio 102 and one camera 104 for use during their shift. Other devices may be acquired as well. As shown in FIG. 1, officer 101 will preferably wear the devices during a shift by attaching the devices to clothing. These devices will form a PAN throughout the officer's shift.

FIG. 2 depicts an example communication system 200 that incorporates PANs created as described above. System 200 includes one or more radio access networks (RANs) 202, a public-safety core network 204, high-speed data network 206, hub (PAN master device) 102, local devices (subordinate devices that serve as smart accessories/sensors) 212, computer 214, and communication links 218, 224, 232, and 234. In a preferred embodiment of the present invention, hub 102 and devices 212 form PAN 240, with communication links 232 between devices 212 and hub 102 taking place utilizing a short-range wireless communication system protocol such as a Bluetooth communication system protocol. Each officer will have their own associated PAN 240. Thus, FIG. 2 illustrates multiple PANs 240 associated with multiple officers.

RAN 202 includes typical RAN elements such as base stations, base station controllers (BSCs), routers, switches, and the like, arranged, connected, and programmed to provide wireless service to user equipment (e.g., hub 102, and the like) in a manner known to those of skill in the relevant art. RAN 202 may implement a direct-mode, conventional, or trunked land mobile radio (LMR) standard or protocol such as European Telecommunications Standards Institute (ETSI) Digital Mobile Radio (DMR), a Project 25 (P25) standard defined by the Association of Public Safety Communications Officials International (APCO), Terrestrial Trunked Radio (TETRA), or other LMR radio protocols or standards.

High-speed data network 206 is provided. Network 206 may comprise a Long Term Evolution (LTE), LTE-Advance, or 5G protocol including multimedia broadcast multicast services (MBMS) or single site point-to-multipoint (SC-PTM) over which an open mobile alliance (OMA) push to talk (PTT) over cellular (OMA-PoC), a voice over IP (VoIP), an LTE Direct or LTE Device to Device, or a PTT over IP (PoIP) application may be implemented. In still further embodiments, network 206 may implement a Wi-Fi protocol perhaps in accordance with an IEEE 802.11 standard (e.g., 802.11a, 802.11b, 802.11g) or a WiMAX protocol perhaps operating in accordance with an IEEE 802.16 standard.

Sensor 212 data that is reported to dispatch center 214 is typically (but not necessarily) accomplished by utilizing network 206, capable of achieving large data rates, while voice communications take place through network 204. Thus, voice communications among public-safety officers typically take place through one network, while data shared among public-safety officers typically take place through another network.

Public-safety core network 204 may include one or more packet-switched networks and/or one or more circuit-switched networks, and in general provides one or more public-safety agencies with any necessary computing and communication needs, transmitting any necessary public-safety-related data and communications.

Hub 102 serves as a PAN master device, and may be any suitable computing and communication device configured to engage in wireless communication with RAN 202 and/or network 206 over the air interface as is known to those in the relevant art. Moreover, one or more hubs 102 are further configured to engage in wired and/or wireless communication with one or more local device 212 via the communication link 232. Hub 102 will be configured to forward information received from PAN devices 212 to, for example, dispatch center 214. The information can be forwarded to the dispatch center via RANs 202 and/or network 206 based on a combination of device 212 inputs. In one embodiment, all information received from sensors 212 will be forwarded to center 214 via RAN 202 or network 206. In another embodiment, hub 102 will filter the information sent, and only send high-priority information back to dispatch center 214. More particularly, sensors 212 may forward data to dispatch center 214 only after a threshold event is detected (e.g., officer is running, gun has been drawn, officer has fell down, . . . , etc.).

It should also be noted that any one or more of the communication links 218, 224, 232, 234 could include one or more wireless-communication links and/or one or more wired-communication links.

In a preferred embodiment, devices 212 and hub 102 comprise any device capable of forming a PAN, although the present invention may be implemented for devices not forming a PAN. Devices 212 may comprise, for example, a gun-draw sensor, a body temperature sensor, an accelerometer, a heart-rate sensor, a breathing-rate sensor, a camera, a man-down sensor, a GPS receiver capable of determining a location, speed, and direction of the user device, smart handcuffs, a clock, calendar, sound detector, environmental sensors (e.g. a thermometer capable of determining an ambient temperature, humidity, presence of dispersed chemicals, radiation detector, electric field detector, magnetic field detector, etc.), an accelerometer, a biometric sensor (e.g., wristband), a barometer, speech recognition circuitry, a gunshot detector, an ambient sound detector . . . , etc. Some examples follow:

A sensor-enabled holster 212 may be provided that maintains and/or provides state information regarding a weapon or other item normally disposed within the user's sensor-enabled holster 212. The sensor-enabled holster 212 may detect a change in state (presence to absence) and/or an action (removal) relative to the weapon normally disposed within the sensor-enabled holster 212. The detected change in state and/or action may be reported to a portable radio acting as hub 102 via its short-range transceiver. In some embodiments, the sensor-enabled holster may also detect whether the first responder's hand is resting on the weapon even if it has not yet been removed from the holster and provide such information to portable radio 102. Other possibilities exist as well. Such sensor-enabled holsters typically comprise a switch that is “pressed” when a gun is inserted into a holster. Removal of the gun causes the switch to activate.

A biometric sensor 212 (e.g., a biometric wristband) may be provided for tracking an activity of the user or a health status of the user 101, and may include one or more movement sensors (such as an accelerometer, magnetometer, and/or gyroscope) that may periodically or intermittently provide to the portable radio (acting as hub 102) indications of orientation, direction, steps, acceleration, and/or speed, and indications of health such as one or more of a captured heart rate, a captured breathing rate, and a captured body temperature of the user 101, perhaps accompanying other information.

An accelerometer 212 may be provided to measures acceleration and provide this information to hub 102. Single and multi-axis models are available to detect magnitude and direction of the acceleration as a vector quantity, and may be used to sense orientation, acceleration, vibration shock, and falling.

A gyroscope 212 may be provided that is capable of measuring orientation based on the principles of conservation of angular momentum. One type of gyroscope, a microelectromechanical system (MEMS) based gyroscope, uses lithographically constructed versions of one or more of a tuning fork, a vibrating wheel, or resonant solid to measure orientation. Other types of gyroscopes could be used as well.

A magnetometer 212 may be provided to measure the strength and/or direction of the magnetic field in the vicinity of the device, and may be used to determine a direction in which a person or device is facing.

A heart rate sensor 212 may be provided and use electrical contacts with the skin to monitor an electrocardiography (EKG) signal of its wearer, or may use infrared light and imaging device to optically detect a pulse rate of its wearer, among other possibilities, and report this information to hub 102.

A breathing rate sensor 212 may be provided to monitor breathing rate and provide this information to hub 102. The breathing rate sensor may include use of a differential capacitive circuits or capacitive transducers to measure chest displacement and thus breathing rates. In other embodiments, a breathing sensor may monitor a periodicity of mouth and/or nose-exhaled air (e.g., using a humidity sensor, temperature sensor, capnometer or spirometer) to detect a respiration rate. Other possibilities exist as well.

A body temperature sensor 212 may be provided, and report temperature to hub 102. Such a sensor includes an electronic digital or analog sensor that measures a skin temperature using, for example, a negative temperature coefficient (NTC) thermistor or a resistive temperature detector (RTD), may include an infrared thermal scanner module, and/or may include an ingestible temperature sensor that transmits an internally measured body temperature via a short range wireless connection, among other possibilities. Temperature sensor 212 may be used on equipment to determine if the equipment is being worn or not. For example, temperature sensor 212 may exist interior to a bullet-proof vest. I the temperature sensor 212 senses a temperature above a predetermined threshold (e.g., 80 degrees), it may be assumed that the vest is being worn by an officer.

Computer 214 comprises, or is part of, a computer-aided-dispatch center (sometimes referred to as an emergency-call center), that may be manned by an operator providing necessary dispatch operations. For example, computer 214 typically comprises a graphical user interface that provides the dispatch operator necessary information about public-safety officers. As discussed above, much of this information originates from devices/sensors 212 providing information to hub 102, which forwards the information to RAN 202/network 206 and ultimately to computer 214.

Expanding on the above, each user of the system may possess a hub with many associated devices forming a PAN. For each user of the system, computer 214 may track the user's current associated PAN devices (sensors 212) along with sensor data for that user. This information may be used to compile a summary for each user as a function of time (e.g., equipment on hand for each user, along with state information for the equipment during various time periods). The information is preferably stored in database 264. This information may be used to determine an activity score for an officer for various time periods throughout their shift.

With the above in mind, computer 214 may also be configured with a natural language processing (NLP) engine configured to determine the intent and/or content of any written report. The NLP engine may also analyze a digital report being written or previously written to determine a content score for various time periods over the officer's shift.

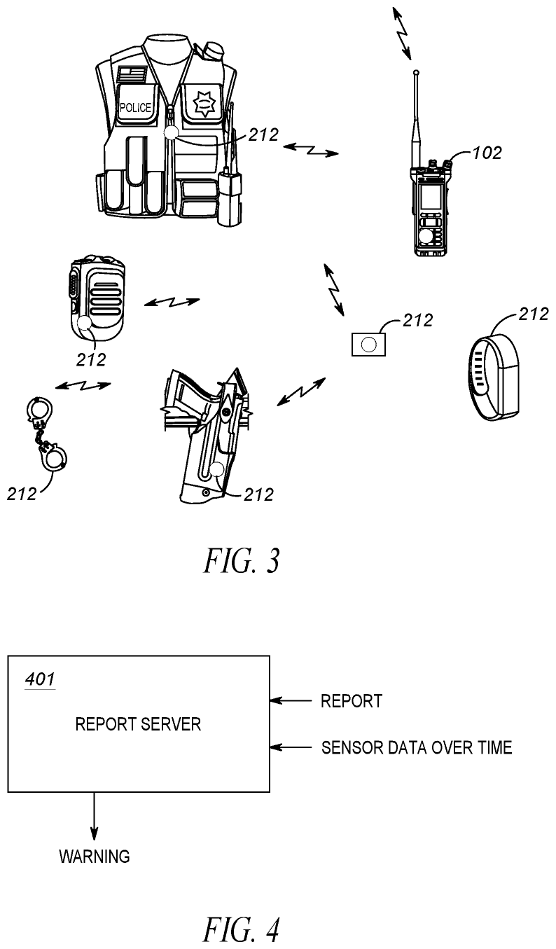

FIG. 3 depicts another view of a personal-area network 240 of FIG. 2. Personal-area network comprises a very local-area network that has a range of, for example 10 feet. As shown in FIG. 3, various devices 212 are that attach to clothing utilized by a public-safety officer. In this particular example, a bio-sensor is located within a police vest, a voice detector is located within a police microphone, smart handcuffs 212 are usually located within a handcuff pouch (not shown), a gun-draw sensor is located within a holster, a smart watch 212 is provided to monitor various biological parameters (e.g., heartrate, blood pressure, . . . , etc.) and a camera 212 is provided.

Devices 212 and hub 102 form a PAN 240. PAN 240 preferably comprises a Bluetooth PAN. Devices 212 and hub 102 are considered Bluetooth devices in that they operate using a Bluetooth, a short range wireless communications technology at the 2.4 GHz band, commercially available from the “Bluetooth special interest group”. Devices 212 and hub 102 are connected via Bluetooth technology in an ad hoc fashion forming a PAN. Hub 102 serves as a master device while devices 212 serve as slave devices.

Hub 102 provides information to the officer, and/or forwards local status alert messages describing each sensor state/trigger event over a wide-area network (e.g., RAN/Core Network) to computer 214. In alternate embodiments of the present invention, hub 102 may forward the local status alerts/updates for each sensor to mobile and non-mobile peers (shift supervisor, peers in the field, etc), or to the public via social media. RAN core network preferably comprises a network that utilizes a public-safety over-the-air protocol. Thus, hub 102 receives sensor data via a first network (e.g., Bluetooth PAN network), and forwards the information to computer 214 via a second network (e.g., a public safety wide area network (WAN) or a high-speed data network (WAN)).

Report server 401 is shown in FIG. 4. Preferably, report server 401 lies within computer 214, however, report server 401 may lie in any piece of network equipment either shown or not shown in FIG. 2. Additionally, report server 401 may be distributed across several pieces of network equipment. Report server 401 is configured to analyze a particular report written by an officer to determine a content score for the report for various time periods covered/described within the report. Report server 401 is also configured to determine an activity score for the officer for time periods covered by the report. Report server 401 is also configured to warn an officer if an activity score for a particular period of time greatly exceeds the content score of the report for the same period of time.

With the above in mind, sensor data is received by server 401 along with a report. An activity score is generated by server 401 for time periods covered by the report, and a content score is generated by server 401 for time periods covered by the report. The time periods may be, for example, 1 hour periods, 30 minute periods, 15 minute periods, 5 minute periods, or any other time period. Based on the activity score and the content score for any particular time period, a user is warned about potential missing details within report for the time period.

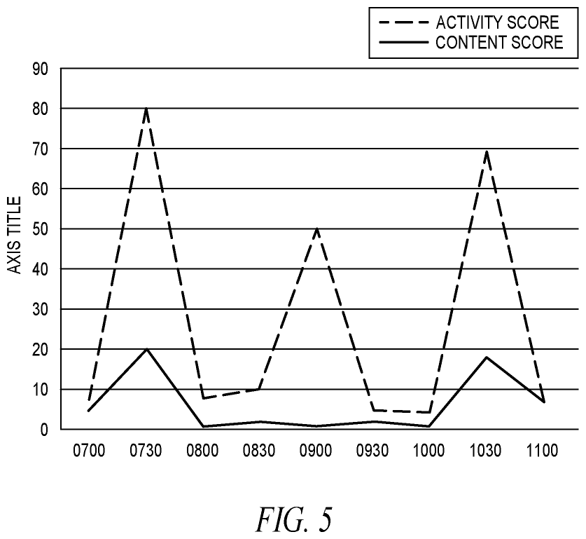

FIG. 5 is an example graph illustrating an activity score and a content score for an officer over time periods 0700 to 1100. As is evident, the activity score and content score vary over those time periods. The time periods are ½ hour time periods. Server 401 may be configured to warn a user if the content score is much lower than the activity score. For example, a warning may be provided to the officer writing the report if the content score is more than X times lower (e.g., 5× lower) than the activity score. If this is the case, then server 401 will warn the report writer that at 0900 there appears to be little content covering that time period (0900-0930). The warning may also comprise a list of sensors 212 that were active during that time period.

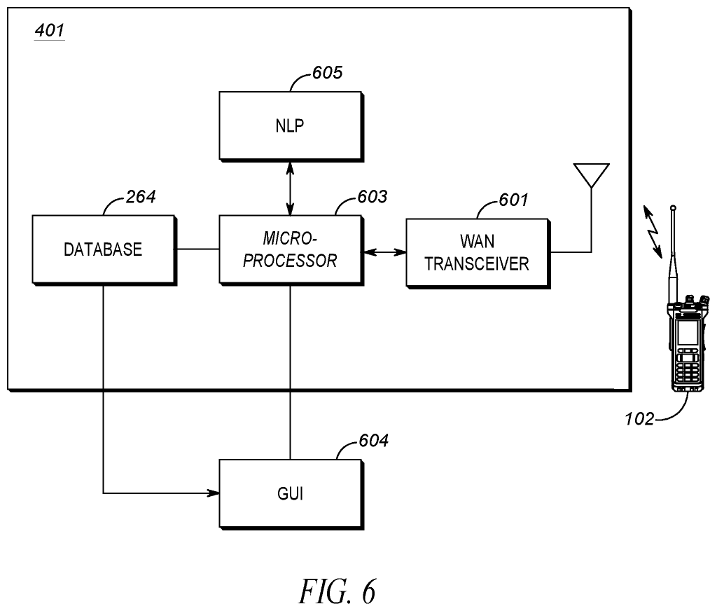

FIG. 6 is a more-detailed block diagram of server 401. In an embodiment, server 401 is embodied within a dispatch center, however in alternate embodiments the device may be embodied within the public-safety core network 204, or more computing devices in a cloud compute cluster (not shown), or some other communication device not illustrated in FIG. 5, and/or may be a distributed communication device across two or more entities.

FIG. 6 shows those components (not all necessary) for server 401 to determine if a warning should be sent to a report writer, wherein the warning notifies the report writer that information may be missing within the report. As shown, server 401 may include a wide-area-network (WAN) transceiver 601 (e.g., a transceiver that utilizes a public-safety communication-system protocol or broadband communication-system protocol), logic circuitry 603, database 264, and network natural-language processor 605. In other implementations, server 401 may include more, fewer, or different components. Regardless, all components are connected via common data busses as known in the art.

WAN transceiver 601 may comprise well known long-range transceivers that utilize any number of network system protocols. (As one of ordinary skill in the art will recognize, a transceiver comprises both a transmitter and a receiver for transmitting and receiving data). For example, WAN transceiver 601 may be operable to utilize a next-generation cellular communications protocol operated by a cellular service provider, or any public-safety protocol such as an APCO 25 network or the FirstNet broadband network. It should be noted that WAN transceiver 601 is shown as part of server 401, however, WAN transceiver 601 may be located in RAN 502, with a direct link to server 401. WAN transceiver 601 receives communications from PAN devices 212 (relayed through hub 102) about their current statuses. This information is stored within database 264 along with a time the communication was received.

GUI 604 provides a man/machine interface for receiving an input from a user and displaying information. For example, GUI 506 may provide a way of conveying (e.g., displaying) information received from logic circuitry 603. Part of this information may comprise a warning that information may be missing from a report. In order to provide the above features (and additional features), GUI 506 may comprise any combination of a touch screen, a computer screen, a keyboard, or any other interface needed to receive a user input and provide information to the user. GUI 604 is also utilized by a user to write a report about an incident. A report template is preferably obtained from database 264, and is filled in by the user/officer when writing the report. The finished report may be stored in database 264. Logic circuitry 603 accesses the finished report, derives a content score based on the text of the report, and warns the user if content is potentially missing from the report.

Logic circuitry 603 comprises a digital signal processor (DSP), general purpose microprocessor, a programmable logic device, or application specific integrated circuit (ASIC) and is operable to determine if a warning should be provided to a report writer and provide the warning to a report writer as described herein. The warning may simply be a message that displays on GUI 604 that potential content is missing from a report.

Database 264 is provided. Database 264 comprises standard memory (such as RAM, ROM, . . . , etc) and serves to store reports on incidents submitted by officers along with sensor data received from PANs.

Finally, natural-language processor (NLP) 605 is provided. NLP 605 comprises well known circuitry that along with logic circuitry 603, analyzes, understands, and derives meaning from human language in a smart and useful way. By utilizing NLP/logic circuitry, automatic summarization, translation, named entity recognition, relationship extraction, sentiment analysis, speech recognition, and topic segmentation can take place on a report to determine various topics discussed within the report for various time periods. For example, NLP/logic circuitry combination may determine that between 0700 and 0800 the report writer discussed five topics.

With the above in mind, the apparatus of FIG. 6 comprises a transceiver 601 configured to receive sensor information regarding sensors associated with a person, a database 264 configured to store the sensor information, and logic circuitry 603 configured to receive a report written by the person, analyze the sensor information to determine an activity level for the person for various time periods, analyze the report to determine an amount of content within the report for the various time periods, determine an activity score for a time period that is based on an activity level for that time period, determine a content score for the time period that is based on an amount of content within the report describing what happened during the time period, and warn the person that content may be missing from the report based on a comparison of the activity score for the time period and the content score for the time period.

As discussed above, the sensor information may comprise sensor information from a sensor part of personal-area network (PAN) associated with the person. Additionally, the logic circuitry may analyze the sensor information to determine an activity level by analyzing the sensor information to determine how many sensors were active during the time period. Additionally, the logic circuitry may analyze the report to determine the amount of content within the report for the time period by analyzing the report to determine how many words were used to describe what happened during the time period. Additionally, the logic circuitry may analyze the report to determine the amount of content within the report for the time period by analyzing the report to determine how many topics were used to describe what happened during the time period. Finally, the logic circuitry may warn the user by sending the user a message on a graphical-user interface (GUI).

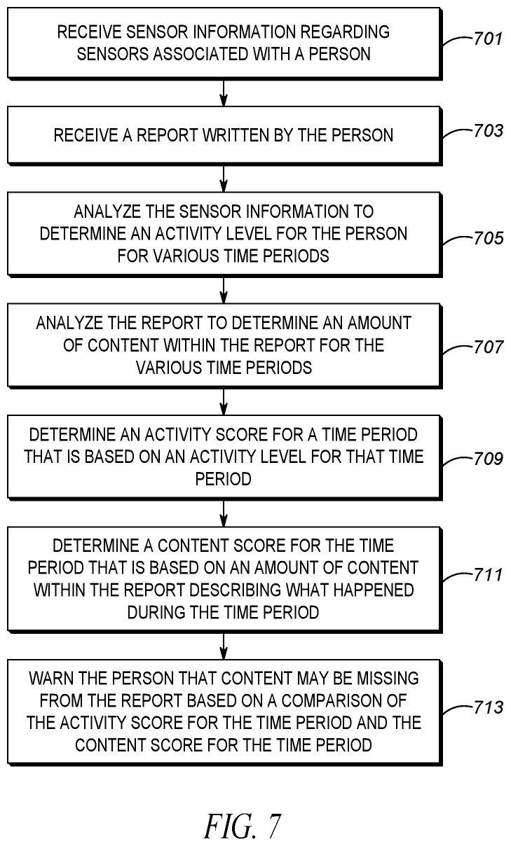

FIG. 7 is a flow chart showing operation of server 401. The logic flow begins at step 701 where WAN transceiver 601 receives sensor information regarding sensors associated with a person, passes this information to logic circuitry 603, which stores the information in database 264. At step 703, logic circuitry 603 receives a report written by the person, wherein the report is preferably received by the person via GUI 604, or alternatively retrieved from database 264. At step 705, logic circuitry 603 analyzes the sensor information to determine an activity level for the person for various time periods, then analyzes the report to determine an amount of content within the report for the various time periods (step 707), determines an activity score for a time period that is based on an activity level for that time period (step 709), and determines a content score for the time period that is based on an amount of content within the report describing what happened during the time period (step 711). Finally, at step 713, logic circuitry 603 warns the person that content may be missing from the report based on a comparison of the activity score for the time period and the content score for the time period.

In one embodiment of the present invention, the step of receiving the sensor information comprises the step of receiving the sensor information from a personal-area network (PAN) associated with the person, via a wide-area network (WAN). In one embodiment of the present invention the step of analyzing the sensor information to determine an activity level comprises the step of analyzing the sensor information to determine how many sensors were active during the time period. In one embodiment of the present invention the step of analyzing the report to determine the amount of content within the report for the time period comprises the step of analyzing the report to determine how many words were used to describe what happened during the time period. In one embodiment of the present invention the step of analyzing the report to determine the amount of content within the report for the time period comprises the step of analyzing the report to determine how many topics were used to describe what happened during the time period. Finally, in one embodiment of the present invention the step of warning the user comprises the step of sending the user a message on a graphical-user interface (GUI).

Those skilled in the art will further recognize that references to specific implementation embodiments such as “circuitry” may equally be accomplished via either on general purpose computing apparatus (e.g., CPU) or specialized processing apparatus (e.g., DSP) executing software instructions stored in non-transitory computer-readable memory. It will also be understood that the terms and expressions used herein have the ordinary technical meaning as is accorded to such terms and expressions by persons skilled in the technical field as set forth above except where different specific meanings have otherwise been set forth herein.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way, but may also be configured in ways that are not listed.

It will be appreciated that some embodiments may be comprised of one or more generic or specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the method and/or apparatus described herein. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used.

Moreover, an embodiment can be implemented as a computer-readable storage medium having computer readable code stored thereon for programming a computer (e.g., comprising a processor) to perform a method as described and claimed herein. Examples of such computer-readable storage mediums include, but are not limited to, a hard disk, a CD-ROM, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

What is claimed is:

1. An apparatus comprising:
a transceiver configured to receive sensor information regarding sensors associated with a person;
a database configured to store the sensor information;
logic circuitry configured to:
receive a report written by the person;
analyze the sensor information to determine an activity level for the person for various time periods;
analyze the report to determine an amount of content within the report for the various time periods;
determine an activity score for a time period that is based on an activity level for that time period;
determine a content score for the time period that is based on an amount of content within the report describing what happened during the time period; and
warn the person that content may be missing from the report based on a comparison of the activity score for the time period and the content score for the time period.

2. The apparatus of claim 1 wherein the sensor information comprises sensor information from a sensor part of personal-area network (PAN) associated with the person.

3. The apparatus of claim 1 wherein the logic circuitry analyzes the sensor information to determine an activity level by analyzing the sensor information to determine how many sensors were active during the time period.

4. The apparatus of claim 1 wherein the logic circuitry analyzes the report to determine the amount of content within the report for the time period by analyzing the report to determine how many words were used to describe what happened during the time period.

5. The apparatus of claim 1 wherein the logic circuitry analyzes the report to determine the amount of content within the report for the time period by analyzing the report to determine how many topics were used to describe what happened during the time period.

6. The apparatus of claim 1 wherein the logic circuitry warns the person by sending the person a message on a graphical-user interface (GUI).

7. A method comprising the steps of:
receiving sensor information regarding sensors associated with a person;
receiving a report written by the person;
analyzing the sensor information to determine an activity level for the person for various time periods;
analyzing the report to determine an amount of content within the report for the various time periods;
determining an activity score for a time period that is based on an activity level for that time period;
determining a content score for the time period that is based on an amount of content within the report describing what happened during the time period; and
warning the person that content may be missing from the report based on a comparison of the activity score for the time period and the content score for the time period.

8. The method of claim 7 wherein the step of receiving the sensor information comprises the step of receiving the sensor information from a personal-area network (PAN) associated with the person, via a wide-area network (WAN).

9. The method of claim 7 wherein the step of analyzing the sensor information to determine an activity level comprises the step of analyzing the sensor information to determine how many sensors were active during the time period.

10. The method of claim 7 wherein the step of analyzing the report to determine the amount of content within the report for the time period comprises the step of analyzing the report to determine how many words were used to describe what happened during the time period.

11. The method of claim 7 wherein the step of analyzing the report to determine the amount of content within the report for the time period comprises the step of analyzing the report to determine how many topics were used to describe what happened during the time period.

12. The method of claim 7 wherein the step of warning the person comprises the step of sending the person a message on a graphical-user interface (GUI).

13. A method comprising the steps of:
receiving sensor information regarding sensors associated with a person;
receiving a report written by the person;
analyzing the sensor information to determine an activity level for the person for various time periods;
analyzing the report to determine an amount of content within the report for the various time periods;
determining an activity score for a time period that is based on an activity level for that time period;
determining a content score for the time period that is based on an amount of content within the report describing what happened during the time period; and
warning the person that content may be missing from the report based on a comparison of the activity score for the time period and the content score for the time period;
wherein the step of receiving the sensor information comprises the step of receiving the sensor information from a personal-area network (PAN) associated with the person, via a wide-area network (WAN);
wherein the step of analyzing the sensor information to determine an activity level comprises the step of analyzing the sensor information to determine how many sensors were active during the time period;
wherein the step of analyzing the report to determine the amount of content within the report for the time period comprises the step of analyzing the report to determine how many words were used to describe what happened during the time period;
wherein the step of analyzing the report to determine the amount of content within the report for the time period comprises the step of analyzing the report to determine how many topics were used to describe what happened during the time period; and
wherein the step of warning the person comprises the step of sending the person a message on a graphical-user interface (GUI).
